(19) United States
(12) Patent Application Publication
(10) Pub. No.: US 2023/0368393 A1
(43) Pub. Date: Nov. 16, 2023

(54) SYSTEM AND METHOD FOR IMPROVING ANNOTATION ACCURACY IN MRI DATA USING MR FINGERPRINTING AND DEEP LEARNING

(71) Applicant: Case Western Reserve University, Cleveland, OH (US)

(72) Inventors: Mark A. Griswold, Shaker Heights, OH (US); Yong Chen, Beachwood, OH (US); Chaitra Badve, Moreland Hills, OH (US); Rasim Boyacioglu, Cleveland Heights, OH (US)

(21) Appl. No.: 18/315,298

(22) Filed: May 10, 2023

Related U.S. Application Data
(60) Provisional application No. 63/340,331, filed on May 10, 2022.

(51) Int. Cl. G06T 7/11; G01R 33/56; A61B 5/055; G06T 7/00; G06T 7/136

(52) U.S. Cl. CPC G06T 7/11; G01R 33/5608; A61B 5/055; G06T 7/0012; G06T 7/136; G06T 2207/10088; G06T 2207/20081; G06T 2207/20221; G06T 2207/30016; G06T 2207/30096

(57) ABSTRACT

The present application provides an automated system and method for improving and generating annotated magnetic resonance (MRI) images based on magnetic resonance fingerprinting (MRF) data. The present disclosure also provides an automated method for generating the automated system for improving annotated MRI images. In some aspects, the method comprises accessing MRF data, MRI data, and images annotated with bulk-pixel labels that identify a tissue class for a group of patients. The annotated images can be used to train a machine learning system based on MRF data. The system can be trained to assign pixel labels to pixels outside the bulk-pixel labels to create an automated system. The automated system provided may be used to determine disease states using MRF data and generate machine-annotated images that include labels that indicate a tissue class. In this way, the present disclosure provides an automated system and method for improving incomplete annotations.

### CROSS-REFERENCE TO RELATED APPLICATIONS

This application is based on, claims priority to, and incorporates herein in its entirety, U.S. Application Ser. No. 63/340,331 filed on May 10, 2022, and entitled “Improving Annotation Accuracy in MRI Data Using MR Fingerprinting and Deep Learning.”

### STATEMENT REGARDING FEDERALLY SPONSORED RESEARCH OR DEVELOPMENT

NA

### BACKGROUND

The present disclosure relates generally to systems and methods for improving annotation accuracy of magnetic resonance imaging (MRI) data using magnetic resonance (MR) fingerprinting and deep learning.

Annotated MRI data with high accuracy is critical for clinical and research purposes. MRI data are typically labeled by experienced radiologists. However, radiologists tend to only label areas where they have high confidence, which leads to incomplete labeling of every suspicious pixel. Thus, the accuracy of human-annotated images will always be limited.

Magnetic resonance fingerprinting (MRF) is an advanced quantitative imaging method that provides high sensitivity to tissue characteristics by simultaneously encoding multiple important tissue properties. MRF can also be used to automatically create tissue segmentation directly from the raw data. However, current MRF methods cannot provide 100% accuracy due to the heterogeneities of the signal, especially associated with abnormal tissues.

Therefore, a need exists to efficiently produce tissue segmentation with higher accuracy and precision.

### SUMMARY

The present disclosure overcomes the drawbacks of previous technologies by providing a deep learning approach that combines the expertise of radiologists provided by manual annotation with the quantitative multiparametric magnetic resonance fingerprinting (MRF) data to improve annotation accuracy of magnetic resonance imaging (MRI) data.

In accordance with one aspect of the present disclosure, a method for creating an automated system for determining disease states and conditions using MRF data and MRI data is provided. The method can include accessing a set of MRF data, MRI data, and annotated images acquired from a group of patients. The annotate images can include bulk-pixel labels that assign bulk pixels to a tissue class, such as a normal tissue class or a pathological tissue class. The method can also include training a machine learning system based on the annotated images and MRF data using a patch-based approach. The machine learning system can be trained to perform a pixel-based analysis of the pixels outside of the bulk-pixel labels, which can generate an automated system for determining disease states and conditions using a pixel-based machine learning system.

In accordance with another aspect of the present disclosure, a method for creating an automated system for generating improved annotations of MRI data of a patient using MRF data is provided. The method can include accessing annotated MRI images for a group of patients that contain a plurality of pixels labeled as abnormal or unlabeled. The method can also include accessing MRF data measured at each of the pixels that was labeled as abnormal for a subset of the group of patients. The method can further include using the MRF data to train a machine learning algorithm that uses the labeled abnormal pixels as ground truth.

In yet another aspect of the present disclosure, an automated system for determining disease states and conditions is provided. The system can use MRF data and MRI data acquired from a patient. The system can include a controller that is configured to receive and reconstruct the MRF and MRI data from a patient. The controller can deliver the reconstructed MRF and MRI data to a trained machine learning system that was trained using MRF data acquired from a group of patients and annotated images acquired from the group of patients that include bulk-pixel labels assigning bulk pixels to a tissue class, such as normal or pathological. The machine learning algorithm can perform a pixel-by-pixel analysis of the reconstructed MRI data to assign each pixel to a tissue class based on the MRF data. The controller can further generate one or more machine-annotated images of the patient that includes pixels with an assigned tissue class.

Also provided herein is a method for generating an automated system for cleaning annotated MRI images using MRF data. The method can include accessing annotated MRI images and corresponding MRF data. The annotated MRI images may be defined by two regions; the first region can include pixels that are labeled as abnormal, and the second region can include unlabeled pixels. The method can further include splitting the annotated imaging into a group of training data and a group of test data and using the training data to train a machine learning algorithm to assign pixel-wise labels to MRI images based on the MRF data. The ground truth used to train the machine learning algorithm may be defined by the first region of the annotated MRI images in the group of training data that includes the pixels labeled as abnormal.

These are but a few, non-limiting examples of aspects of the present disclosures. Other features, aspects and implementation details will be described hereinafter.

### BRIEF DESCRIPTION OF THE DRAWINGS

The present invention will hereafter be described with reference to the accompanying drawings, wherein like reference numerals denote like elements.

FIG. 1 is a flowchart setting forth steps of a process for cleaning image annotations, in accordance with aspects of the present disclosure.

FIG. 2 is a flowchart setting forth steps of a process for generating image annotations, in accordance with aspects of the present disclosure.

FIG. 3A shows an example of manual and model-generated image annotations for a patient with high-grade glioma.

FIG. 3B shows an example of manual and model-generated image annotations for a patient with low-grade glioma.

FIG. 4A shows an example of manual and model-generated image annotations and probability maps for abnormal tissue types.

FIG. 4B shows an example of the total number of pixels assigned with each tissue type before and after cleaning the annotations with an automated annotation system.

FIG. 5 shows an example of magnetic resonance fingerprinting (MRF) and clinical magnetic resonance imaging (MRI) data that can be used in the process laid out in FIG. 1 to generate cleaned annotations.

FIG. 6 is a block diagram of an example MRI system that can implement the methods described in the present disclosure.

FIG. 7 is a block diagram of an example automated annotation system that can implement the methods of the present disclosure, including generating and using an automated system for cleaning annotated MRI images using MRF data.

FIG. 8 is a block diagram of example components that can implement the system of FIG. 7.

### DETAILED DESCRIPTION

To overcome aforementioned drawbacks, the present disclosure trains a machine learning algorithm to generate cleaned annotations based on manually annotated images and magnetic resonance fingerprinting (MRF) data. The present approach produces annotated images with higher accuracy than using manual labeling or automatic labeling based on MRF data alone. As will be described, the deep learning approach was tested with in vivo brain data to annotate regions of glioma. However, the present approach may be broadly applicable for annotating other normal tissue types or pathologies for other organ systems, as well.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

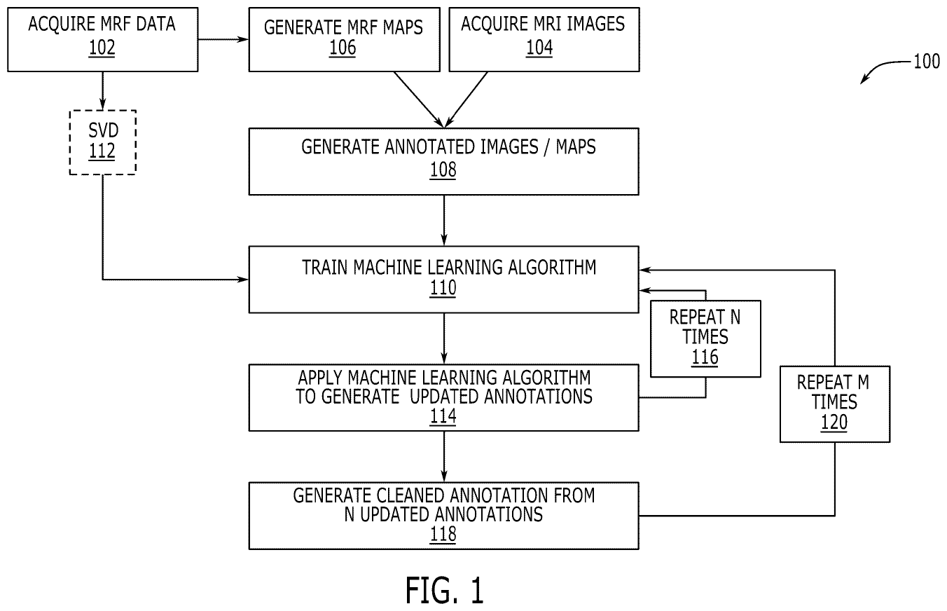

FIG. 1 lays out the general steps of the process 100, in which MRF data and MRI images from a group of subjects or patients can be used to train a machine learning algorithm to generate cleaned annotated images. The process 100 can create an automated system for determining disease states and conditions using the MRF and MRI data. The process 100 begins by acquiring MRF data or group MRF data in block 102 from a group of subjects and acquiring corresponding MRI images in block 104 from the group of subjects. For example, the group of subjects may comprise N=43 patients with known brain tumors. As a non-limiting example, the MRF data of block 102 may include non-contrast 3D fast imaging with steady-state free precession (FISP) whole-brain images of each subject for 1440 time points at which the imaging parameters (e.g., repetition time (TR), echo time (TE), flip angle, etc.) are randomly, pseudo-randomly, or otherwise adjusted. The MRI images of block 104 may include typical clinical whole-brain images of the subjects, such as magnetization prepared rapid gradient echo (MP-RAGE), fluid-attenuated inversion recovery (FLAIR), post-contrast T₁-weighted images, etc. Blocks 102 and 104 may include directly acquiring such data using an MRI system or accessing the stored data by a computer system. Blocks 102 and 104 may also include reconstructing raw MRF and raw MRI data to produce MRF images and MRI images.

In block 106, the MRF data may be used to generate parameter maps. For example, T₁, T₂, proton density, etc. may be estimated for each pixel by pattern matching of the acquired MRF time course with an MRF dictionary. Other MRF fitting techniques may be used to generate MRF maps, such as deep learning approaches.

In block 108 initial annotated images can be produced or accessed for each subject. The annotated images may include bulk-pixel labels that assign bulk pixels to a tissue class, such as normal, benign, pathological, or abnormal. As a non-limiting example, abnormal tissue type classes may broadly include any abnormal tissue types, or may specifically include high-grade glioma, low-grade glioma, peritumor white matter (PWM), and necrosis (NEC). Segmentation may also be applied to identify regions of normal tissue.

The annotated images may be generated by manual segmentation in which regions of interest are manually drawn to identify classes of tissue types. The annotation may be completed by an expert, such as a radiologist or other trained personnel. For example, a radiologist may manually annotate the images based on some combination of the clinical anatomical images, quantitative MRF maps, MRF-based synthetic images, histopathological data, etc. The annotated images may include bulk-pixel labels for pathological tissue, normal tissue, other label types, or some combination thereof. The annotated images may also include unlabeled pixels. The annotated images may also be generated using an automatic technique. For example, partial volume analysis may be used to label white matter (WM), gray matter (GM), and cerebrospinal fluid (CSF).

Advantageously, the annotated images need not be fully annotated. In other words, not every pixel of the annotated images must have a pixel label. Moreover, not every pathological pixel must be labeled as such. For example, the annotated images may include a label for a subset of the pixels containing pathological tissue. In a non-limiting example, the subset may be a set of pixels that includes the pixels for which the expert annotator is highly confident contain pathological tissues. In this example, the annotator may intentionally omit pixels for which they are unsure of the status of the pixel. In another non-limiting example, the annotator may perform an annotation using a convenient shape or lower resolution. For example, the annotator may place a circular or elliptical region of interest in the center of the pathology without taking the time to label each pixel in the non-elliptical border of the pathological region. Thus, it may be assumed that the pixels labeled as pathology (e.g. high-grade glioma, low-grade glioma, PWM, necrosis) in block 108 are correctly labeled. In this way, the pathology labels of block 108 can be used as a reference for training a machine learning algorithm in process block 110 that will correct the normal tissue labels given in block 108 or the unlabeled pixels in block 108 in subsequent steps of process 100 (e.g. block 114). For example, the machine learning algorithm in process block 110 may be used to label the pixels for which the annotator was uncertain or may provide a detailed annotation of the unlabeled non-elliptical border of the pathological ROI.

To train the machine learning algorithm in block 110, the input may include the reference annotated images from block 108 and the MRF data from block 102. Optionally, the MRF data from block 102 may be processed by singular value decomposition (SVD) or another compression method in block 112 in order to reduce the number of network parameters. For example, a time course of 1440 MRF data points for each pixel may be reduced by SVD to be represented as 25 singular values in block 112.

In process block 110, a machine learning algorithm is trained to clean the image annotations, which may include supplementing or correcting the labels of the annotated images. For example, the machine learning algorithm may comprise a convolutional neural network using a U-Net architecture. The data can be split into training data and validation data. In one non-limiting example, to split the data into training and validation data, each patient may be assigned to one of k groups. A subset of the k groups (e.g., k−1) may be used as training data while the remaining groups (e.g., one group) may be used as validation data. For example, ten-fold cross validation may be used in which the patients are split into ten groups; nine groups are used for training, and one is used for validation. As a non-limiting example, the patients may be randomly or pseudo-randomly split into training and validation groups.

The machine learning algorithm may be trained pixelwise to label each pixel in the image. The machine learning algorithm may account for MRF data and use the pixel labels of the annotated images as ground truth. The machine learning algorithm may use a 1D, 2D, or 3D patch-based approach in which the algorithm is trained for each pixel using MRF data from pixels within a defined patch of pixels. In a non-limiting example, the patch may be a 1×1×1 patch in which each pixel may be labeled based on the MRF data acquired at the given pixel. In another non-limiting example, the patch may be larger than 1×1×1 in order to utilize spatial correlation information, including the MRF data from nearby pixels. For example, for a given pixel, the algorithm may determine the pixel label based on MRF data from a 32 pixel×32 pixel patch surrounding the pixel using the pathology labels of the training set as ground truth.

The patch may be of any desired size and is not required to be equal for each pixel. For example, the patch size may be reduced for pixels at the edge of the field of view or for pixels at the edge of the anatomy of interest where the neighboring pixels have low signal. The patch may also be weighted by weights that are learned by the machine learning framework. Additionally or alternatively, the weights may be predefined. For example, the algorithm may include MRF data from neighboring pixels with a weight that decreases with increasing distance from the pixel of interest. As another example, the weight may decrease with increasing noise of the neighboring pixel. The algorithm may further include labels based on expert-annotated images for a patch surrounding the pixel to inform the model, if desired. The labels within the patch may also be weighted, as described above.

The trained machine learning algorithm may be applied in block 114. The algorithm may be applied to data included in the training set, to a second group of annotated data that was not included in the training set (e.g., the validation set), or to a new group of data that is not yet labeled. For example, the machine learning algorithm may be applied to annotated images in order to supplement or update the annotated labels from block 108. The trained machine learning algorithm may be applied to a subset of pixels in the images. For example, the algorithm may be applied only to the pixels that were unlabeled in the annotated images, such as pixels proximate to the bulk-pixel labels. In this way, the bulk-pixel labels can be combined with the machine-generated labels to create a composite image that provides a cleaned annotated image. Additionally or alternatively, the algorithm may be applied to any pixels originally labeled as normal tissue. For example, each pixel labeled as normal tissue (e.g., GM, WM, CBS) may be reassigned a label based on the machine learning algorithm, which may be the same label or a new abnormal label. These cleaned labels can be combined with the abnormal labels provided by the manual annotation to produce a fully annotated image with improved accuracy in the relabeled region. The trained machine learning algorithm may also be applied to all of the pixels or some other subset of pixels in the reference annotated images, if desired.

In block 114, the trained machine learning algorithm may analyze each pixel using MRF data at that pixel. The algorithm may further include MRF data from a patch of surrounding pixels to account for spatial correlation information, as previously described.

The training/testing process may be repeated N times in block 116. In repeating the training/testing process, the groups (e.g., k groups) may be reassigned as training or validation data. Alternatively, the data can be re-split into training and validation sets with each repetition. For example, the training may be repeated 5 times. Each time, the patients can be randomly or pseudo-randomly or otherwise reassigned into ten groups, of which nine can be randomly chosen for training and the remaining one can be used for validation.

After repeating N times, the machine learning algorithm will have generated N updated annotations. In block 118, the N updated annotations can be combined to generate cleaned annotations. For example, a probability map may be generated in block 118 that assigns each pixel with a probability of each tissue class based on the N updated annotations. In a non-limiting example, the probability map may be based on the number of repetitions a pixel was labeled as a given tissue class of the N total repetitions. For example, if a given pixel was labeled as abnormal in four of the N=5 repetitions, the probability may be assigned to 80% abnormal for that pixel.

A final classification can be determined by setting a threshold for the probability map. For example, the threshold may be set to 50%. For example, for each pixel, if it was identified as abnormal for more than 50% of the N repetitions (e.g., at least 3 times out of the 5 trainings), the final classification can be re-assigned as abnormal tissue. Another threshold may also be chosen to balance sensitivity and specificity, for example a threshold of at least 80% may be used. Using a threshold, the probability map may be represented as a binary or multiclass annotated image. For example, pixels containing normal tissue may be represented by 0s, and pixels containing abnormal tissue may be represented by 1s. The type of abnormal tissue class can be determined based on the class with the highest probability from the N repetitions. In this case, for example, normal tissue may be represented by 0s, and low- and high-grade glioma may be represented by 1s and 2s, respectively. Other multi-class classification maps may be used as well, to represent each possible tissue class within the annotated image.

In block 118, generating cleaned annotations may be limited to evaluating only the pixels that were unlabeled by the manual annotation of process block 108. In this way, it can be assumed that the manual annotation is correct for every labeled pixel. In other words, it can be assumed that the radiologist only labeled abnormal pixels, for example, for which they were 100% confident. The manual annotation can then be combined with the machine-generated annotations. In this non-limiting example, the resulting cleaned annotations can include pixels labeled in two ways. The pixels labeled in the reference annotated images (i.e., bulk pixels) can maintain their original labels. The pixels outside of or proximate to the bulk pixels can receive a label based on the probability map.

The training pipeline can be optionally repeated M times in block 120 to iteratively improve the training input annotations. For example, the cleaned annotations from process block 118 can be used as the annotated images to train the network in process block 110, replacing the reference annotated images (e.g., manually annotated images) from block 108 as training data.

Block 118 provides an automated system for determining disease states and conditions using MRF data that can be used to clean image annotations, correct image annotations, or create new image annotations. They system can perform a pixel-by pixel analysis to label reconstructed MRI images, and annotations can be superimposed onto images for viewing and interpretation. The labels can include any number of tissue classes, such as abnormal, normal, benign, pathological, etc. The tissue classes may also include more specific types of normal or abnormal tissues, such as white matter, gray matter, high-grade or low-grade glioblastoma, peritumor white matter, necrosis, etc.

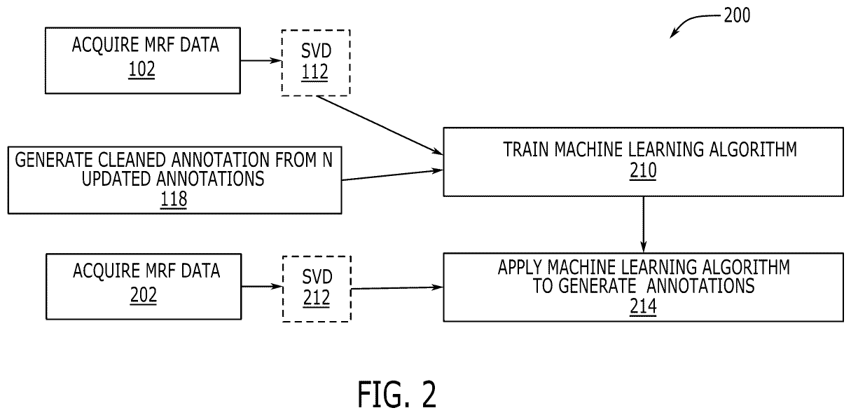

Now referring to FIG. 2, the process can be further expanded for application to new data in the process 200. For example, the cleaned annotations from process block 118 can be used to train a machine learning algorithm to create annotations in block 210. This training process can use the original MRF data of block 102, which can optionally be compressed, such as by SVD, in block 112. The training process can use the cleaned annotations from block 118 as the ground truth. For example, the ground truth annotations may be generated in process 100 using data acquired in a research setting or previously acquired in a clinical setting.

The machine learning algorithm trained in block 210 may comprise a convolutional neural network using a U-Net architecture. It may also include a 1D, 2D, or 3D patch-based approach to account for spatial information. For example, for each given pixel, the model may include a surrounding 3D patch of 32 pixels×32 pixels×32 pixels of the compressed MRF data.

After training the machine learning algorithm in block 210, the algorithm can be applied in block 214 to new data, such as in a clinical setting. For example, MRF data may be acquired in block 202 and optionally compressed in block 212 by SVD or another compression. The trained algorithm can be applied in block 214 to generate annotations without the need for manual input from a radiologist.

By combining processes 100 and 200, radiologists or other trained professionals can quickly annotate a relatively small set of images. This annotation can be done quickly, as the annotator is only required to choose pixels for which they are fully confident are abnormal or another desired tissue class. They may not be required to carefully annotate complex regions, such as spiculated borders, or confirm their annotations in all three planes. These annotations can be cleaned in process 100 to further include pixels for which the annotator was not fully confident were abnormal and to include finer details, like abnormal borders, etc. The cleaned annotations can then be used in process 200 to fully automate annotation of new data, such as clinical data.

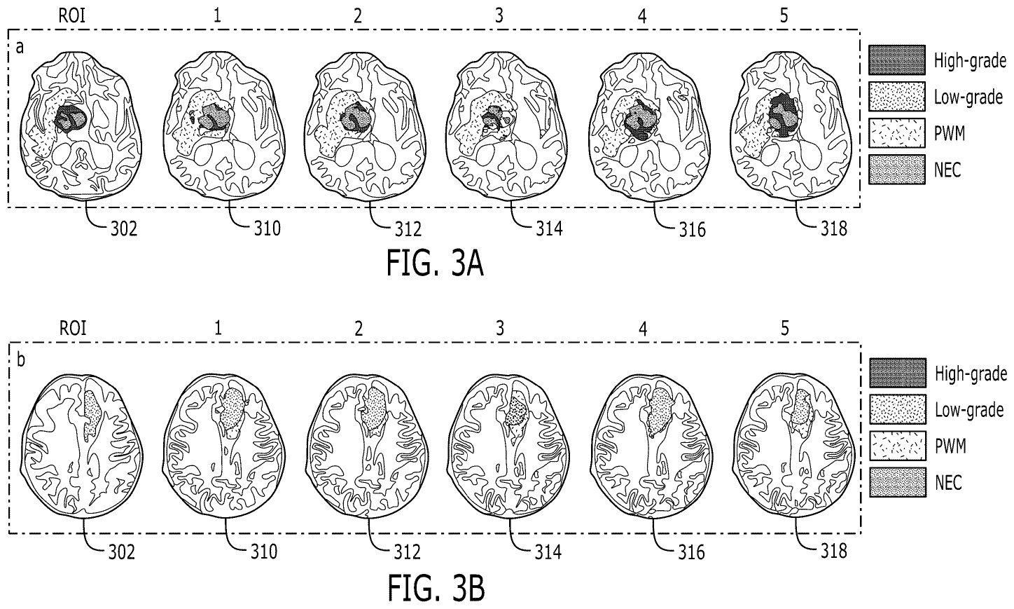

Referring now to FIGS. 3A and 3B, a non-limiting example of the process 100 was applied in the context of brain imaging. FIG. 3A shows a patient with known high-grade glioma, and FIG. 3B shows a patient with known low-grade glioma. The images were initially annotated by a radiologist, as shown in block 108, for example. The manual annotations 302 include labels for high-grade glioma, low-grade glioma, peritumor white matter (PWM), and necrosis (NEC). These annotations 302 were used as part of the ground truth training data to train a machine learning algorithm, such as in block 110. The algorithm was then applied, such as in block 114, which generated an updated annotation 310. The process was repeated such that model was trained and applied a total of N=5 times, generating the updated annotations 310, 312, 314, 316, and 318.

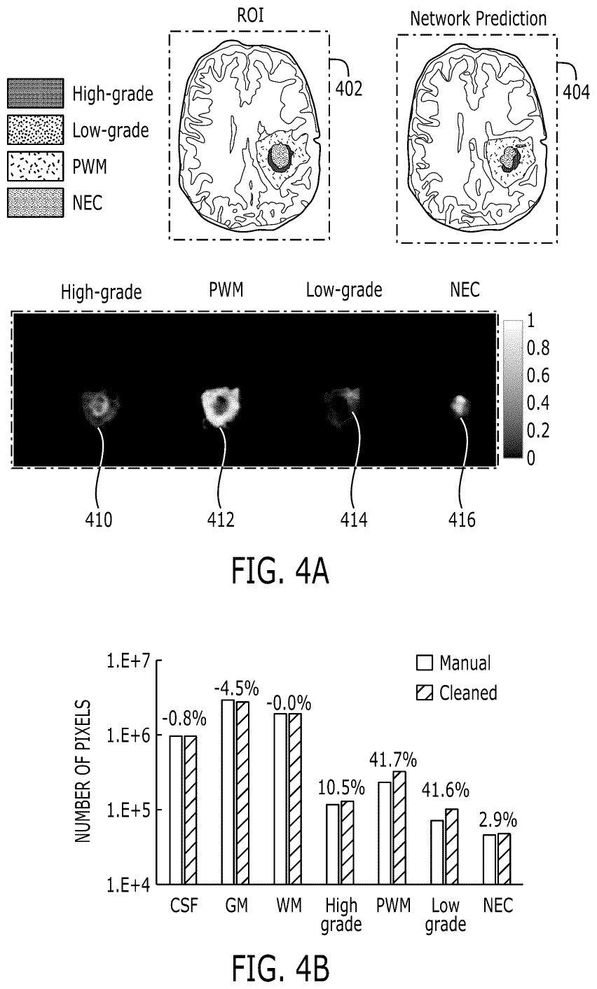

Another example is shown in FIG. 4A for a patient with high-grade glioma. The manual annotation generated an annotation 402, which was used as training data for the algorithm. The machine learning algorithm was trained and applied a total of 5 times to generate 5 updated annotations (e.g., 404). By aggregating the 5 annotations, as described in block 118, for example, a probability was assigned for each tissue type at each pixel. The probabilities were determined based on the number of repetitions (of N) that a pixel was labeled as high-grade (e.g., 410), low-grade glioma (e.g., 412), peritumor white matter (e.g., 414), or necrosis (e.g., 416). For example, if a given pixel was labeled as high-grade glioma three of the N=5 repetitions, the probability of high-grade glioma at that pixel is considered to be 0.6 or 60%. Cleaned annotations were generated based on the probability maps (e.g., 410, 412, 414, and 416) and a predetermined threshold, assigning the tissue type based on the highest probability scores. These cleaned annotations could be further used to train a machine learning algorithm (e.g., block 210), which could be applied to generate accurate annotations from new data, as done, for example, in block 214.

The overall results for an example set of 43 glioblastoma patients are shown in FIG. 4B. The number of pixels assigned to each tissue type is plotted based on manual annotations and annotations cleaned by the automated process 100, as shown in FIG. 4A. Many pixels that were previously identified as grey matter were cleaned by reassigning them as abnormal tissue types, which leads to substantial increases in all four abnormal tissue classes.

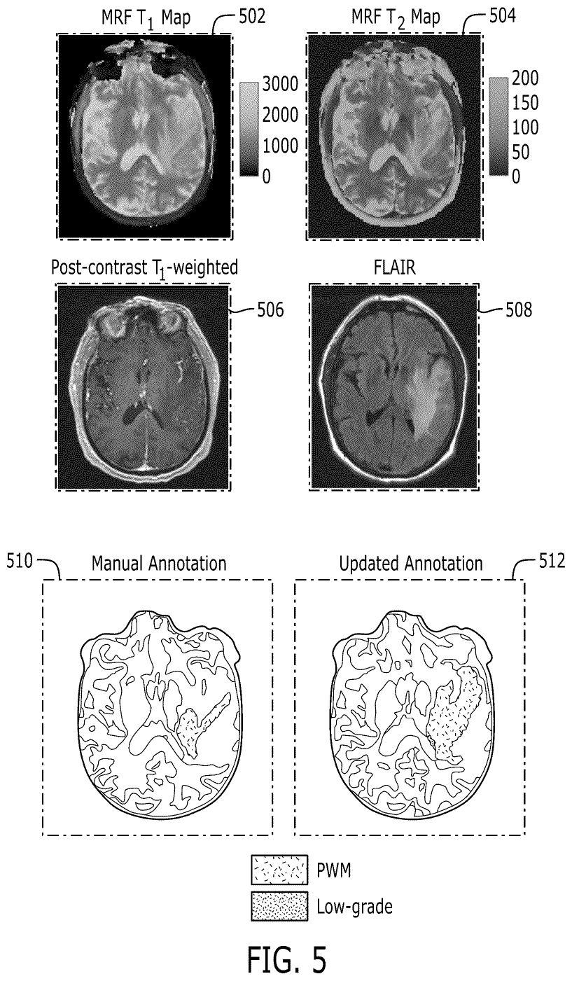

FIG. 5 shows another example of cleaning manual annotations, using process 100, for example. MRF data acquired in block 102 was used to generate T₁ 502 and T₂ 504 maps in block 106. Standard clinical images, including post-contrast T₁-weighted 506 and FLAIR 508 images were acquired in block 104. The MRF maps (i.e., 502 and 504) were used with the clinical images (i.e., 506 and 508) to manually generate annotated images 510 in block 108. The MRF time course data was also compressed using SVD in block 112. After training and applying the machine learning algorithm in blocks 110 and 114, respectively, a total of N=5 times, the updated annotation 512 was generated in block 118. The updated annotation 512 includes an expanded region of peritumor white matter compared to the original manual annotation 510, which matches the MRF T₁ map 502, MRF T₂ map 504, post-contrast T₁-weighted image 506, and FLAIR image 508.

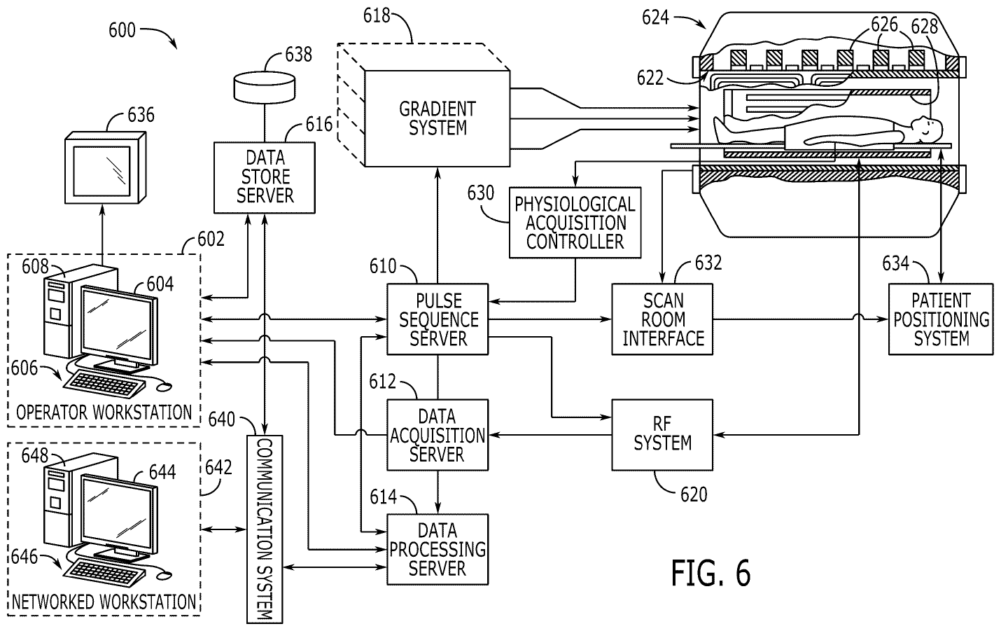

Referring particularly now to FIG. 6, an example of an MRI system 600 that can implement the methods described herein is illustrated. The MRI system 600 includes an operator workstation 602 that may include a display 604, one or more input devices 606 (e.g., a keyboard, a mouse), and a processor 608. The processor 608 may include a commercially available programmable machine running a commercially available operating system. The operator workstation 602 provides an operator interface that facilitates entering scan parameters into the MRI system 600. The operator workstation 602 may be coupled to different servers, including, for example, a pulse sequence server 610, a data acquisition server 612, a data processing server 614, and a data store server 616. The operator workstation 602 and the servers 610, 612, 614, and 616 may be connected via a communication system 640, which may include wired or wireless network connections.

The MRI system 600 also includes a magnet assembly 624 that includes a polarizing magnet 626, which may be a low-field magnet. The MRI system 500 may optionally include a whole-body RF coil 628 and a gradient system 618 that controls a gradient coil assembly 622.

The pulse sequence server 610 functions in response to instructions provided by the operator workstation 602 to operate a gradient system 618 and a radiofrequency (“RF”) system 620. Gradient waveforms for performing a prescribed scan are produced and applied to the gradient system 618, which then excited gradient coils in an assembly 622 to produce the magnetic field gradients (e.g., Gₓ, G_{y}, and G_{z}) that can be used for spatially encoding magnetic resonance signals. The gradient coil assembly 622 forms part of a magnet assembly 624 that includes a polarizing magnet 626 and a whole-body RF coil 628.

RF waveforms are applied by the RF system 620 to the RF coil 628, or a separate local coil to perform the prescribed magnetic resonance pulse sequence. Responsive magnetic resonance signals detected by the RF coil 628, or a separate local coil, are received by the RF system 620. The responsive magnetic resonance signals may be amplified, demodulated, filtered, and digitized under direction of commands produced by the pulse sequence server 610. The RF system 620 includes an RF transmitter for producing a wide variety of RF pulses used in MRI pulse sequences. The RF transmitter is responsive to the prescribed scan and direction from the pulse sequence server 610 to produce RF pulses of the desired frequency, phase, and pulse amplitude waveform. The generated RF pulses may be applied to the whole-body RF coil 628 or to one or more local coils or coil arrays.

The RF system 620 also includes one or more RF receiver channels. An RF receiver channel includes an RF preamplifier that amplifies the magnetic resonance signal received by the coil 628 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

M=\sqrt{(I²+Q²)}

and the phase of the received magnetic resonance signal may also be determined according to the following relationship:

$$ϕ = {tan}^{- 1} ( \frac{Q}{I} )$$

The pulse sequence server 610 may receive patient data from a physiological acquisition controller 630. By way of example, the physiological acquisition controller 630 may receive signals from a number of different sensors connected to the patient, including electrocardiograph (“ECG”) signals from electrodes, or respiratory signals from a respiratory bellows or other respiratory monitoring devices. These signals may be used by the pulse sequence server 610 to synchronize, or “gate,” the performance of the scan with the subject's heartbeat or respiration.

The pulse sequence server 610 may also connect to a scan room interface circuit 632 that receives signals from various sensors associated with the condition of the patient and the magnet system. Through the scan room interface circuit 632, a patient positioning system 634 can receive commands to move the patient to desired positions during the scan.

The digitized magnetic resonance signal samples produced by the RF system 620 are received by the data acquisition server 612. The data acquisition server 612 operates in response to instructions downloaded from the operator workstation 602 to receive the real-time magnetic resonance data and provide buffer storage, so that data are not lost by data overrun. In some scans, the data acquisition server 612 passes the acquired magnetic resonance data to the data processor server 614. In scans that require information derived from acquired magnetic resonance data to control the further performance of the scan, the data acquisition server 612 may be programmed to produce such information and convey it to the pulse sequence server 610. For example, during pre-scans, magnetic resonance data may be acquired and used to calibrate the pulse sequence performed by the pulse sequence server 610. As another example, navigator signals may be acquired and used to adjust the operating parameters of the RF system 620 or the gradient system 618, or to control the view order in which k-space is sampled. In still another example, the data acquisition server 612 may also process magnetic resonance signals used to detect the arrival of a contrast agent in a magnetic resonance angiography (“MRA”) scan. For example, the data acquisition server 612 may acquire magnetic resonance data and processes it in real-time to produce information that is used to control the scan.

The data processing server 614 receives magnetic resonance data from the data acquisition server 612 and processes the magnetic resonance data in accordance with instructions provided by the operator workstation 602. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 614 are conveyed back to the operator workstation 602 for storage. Real-time images may be stored in a data base memory cache, from which they may be output to operator display 602 or a display 636. Batch mode images or selected real time images may be stored in a host database on disc storage 638. When such images have been reconstructed and transferred to storage, the data processing server 614 may notify the data store server 616 on the operator workstation 602. The operator workstation 602 may be used by an operator to archive the images, produce films, or send the images via a network to other facilities.

The MRI system 600 may also include one or more networked workstations 642. For example, a networked workstation 642 may include a display 644, one or more input devices 646 (e.g., a keyboard, a mouse), and a processor 648. The networked workstation 642 may be located within the same facility as the operator workstation 602, or in a different facility, such as a different healthcare institution or clinic.

The networked workstation 642 may gain remote access to the data processing server 614 or data store server 616 via the communication system 640. Accordingly, multiple networked workstations 642 may have access to the data processing server 614 and the data store server 616. In this manner, magnetic resonance data, reconstructed images, or other data may be exchanged between the data processing server 614 or the data store server 616 and the networked workstations 642, such that the data or images may be remotely processed by a networked workstation 642.

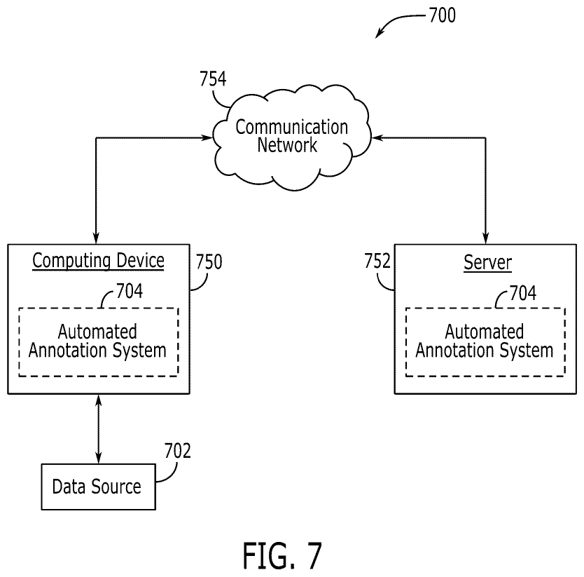

Referring now to FIG. 7, an example of an MRI system 700 is shown, which may be used in accordance with some aspects of the systems and methods described in the present disclosure. As shown in FIG. 7, a computing device 750 can receive one or more types of data (e.g., signal evolution data, k-space data, receiver coil sensitivity data) from data source 702. In some configurations, computing device 750 can execute at least a portion of an automated annotation system 704 to reconstruct images from magnetic resonance data (e.g., k-space data) acquired using an MRF or other technique. In some configurations, the automated annotation system 704 can implement an automated pipeline to provide annotated MRI images, MRF maps, MRF synthetic images, etc.

Additionally or alternatively, in some configurations, the computing device 750 can communicate information about data received from the data source 702 to a server 752 over a communication network 754, which can execute at least a portion of the automated annotation system 704. In such configurations, the server 752 can return information to the computing device 750 (and/or any other suitable computing device) indicative of an output of the automated annotation system 704.

In some configurations, computing device 750 and/or server 752 can be any suitable computing device or combination of devices, such as a desktop computer, a laptop computer, a smartphone, a tablet computer, a wearable computer, a server computer, a virtual machine being executed by a physical computing device, and so on. The computing device 750 and/or server 752 can also reconstruct images from the data.

In some configurations, data source 702 can be any suitable source of data (e.g., measurement data, images reconstructed from measurement data, processed image data), such as an MRI system, another computing device (e.g., a server storing measurement data, images reconstructed from measurement data, processed image data), and so on. In some configurations, data source 702 can be local to computing device 750. For example, data source 702 can be incorporated with computing device 750 (e.g., computing device 750 can be configured as part of a device for measuring, recording, estimating, acquiring, or otherwise collecting or storing data). As another example, data source 702 can be connected to computing device 750 by a cable, a direct wireless link, and so on. Additionally or alternatively, in some configurations, data source 702 can be located locally and/or remotely from computing device 750, and can communicate data to computing device 750 (and/or server 752) via a communication network (e.g., communication network 754).

In some configurations, communication network 754 can be any suitable communication network or combination of communication networks. For example, communication network 754 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some configurations, communication network 754 can be a local area network, a wide area network, a public network (e.g., the Internet), a private or semi-private network (e.g., a corporate or university intranet), any other suitable type of network, or any suitable combination of networks. Communications links shown in FIG. 7 can each be any suitable communications link or combination of communications links, such as wired links, fiber optic links, Wi-Fi links, Bluetooth links, cellular links, and so on.

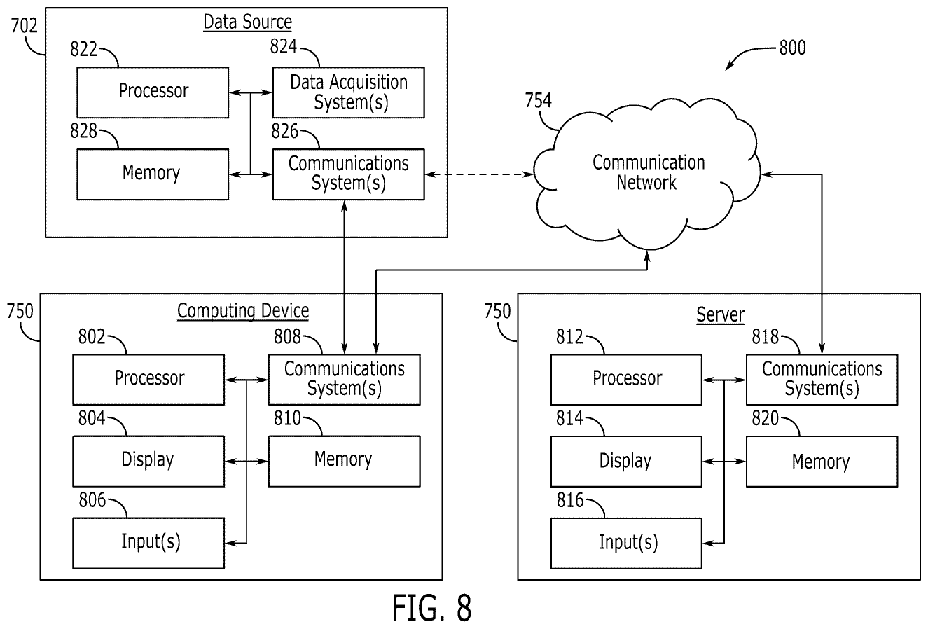

Referring now to FIG. 8, an example of hardware 800 that can be used to implement data source 702, computing device 750, and server 752 in accordance with some configurations of the systems and methods described in the present disclosure is shown.

As shown in FIG. 8, in some configurations, computing device 750 can include a processor 802, a display 804, one or more inputs 806, one or more communication systems 808, and/or memory 810. In some configurations, processor 802 can be any suitable hardware processor or combination of processors, such as a central processing unit (“CPU”), a graphics processing unit (“GPU”), and so on. In some configurations, display 804 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some configurations, inputs 806 can include any suitable input devices and/or sensors that can be used to receive user input, such as a keyboard, a mouse, a touchscreen, a microphone, and so on.

In some configurations, communications systems 808 can include any suitable hardware, firmware, and/or software for communicating information over communication network 754 and/or any other suitable communication networks. For example, communications systems 808 can include one or more transceivers, one or more communication chips and/or chip sets, and so on. In a more particular example, communications systems 808 can include hardware, firmware, and/or software that can be used to establish a Wi-Fi connection, a Bluetooth connection, a cellular connection, an Ethernet connection, and so on.

In some configurations, memory 810 can include any suitable storage device or devices that can be used to store instructions, values, data, or the like, that can be used, for example, by processor 802 to present content using display 804, to communicate with server 752 via communications system(s) 808, and so on. Memory 810 can include any suitable volatile memory, non-volatile memory, storage, or any suitable combination thereof. For example, memory 810 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some configurations, memory 810 can have encoded thereon, or otherwise stored therein, a computer program for controlling operation of computing device 750. In such configurations, processor 802 can execute at least a portion of the computer program to present content (e.g., images, user interfaces, graphics, tables), receive content from server 752, transmit information to server 752, and so on. For example, the processor 802 and the memory 810 can be configured to perform the methods described herein.

In some configurations, server 752 can include a processor 812, a display 814, one or more inputs 816, one or more communications systems 818, and/or memory 820. In some configurations, processor 812 can be any suitable hardware processor or combination of processors, such as a CPU, a GPU, and so on. In some configurations, display 814 can include any suitable display devices, such as an LCD screen, LED display, OLED display, electrophoretic display, a computer monitor, a touchscreen, a television, and so on. In some configurations, inputs 816 can include any suitable input devices and/or sensors that can be used to receive user input, such as a keyboard, a mouse, a touchscreen, a microphone, and so on.

In some configurations, communications systems 818 can include any suitable hardware, firmware, and/or software for communicating information over communication network 754 and/or any other suitable communication networks. For example, communications systems 818 can include one or more transceivers, one or more communication chips and/or chip sets, and so on. In a more particular example, communications systems 818 can include hardware, firmware, and/or software that can be used to establish a Wi-Fi connection, a Bluetooth connection, a cellular connection, an Ethernet connection, and so on.

In some configurations, memory 820 can include any suitable storage device or devices that can be used to store instructions, values, data, or the like, that can be used, for example, by processor 812 to present content using display 814, to communicate with one or more computing devices 750, and so on. Memory 820 can include any suitable volatile memory, non-volatile memory, storage, or any suitable combination thereof. For example, memory 820 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 820 can have encoded thereon a server program for controlling operation of server 752. In such configurations, processor 812 can execute at least a portion of the server program to transmit information and/or content (e.g., data, images, a user interface) to one or more computing devices 750, receive information and/or content from one or more computing devices 750, receive instructions from one or more devices (e.g., a personal computer, a laptop computer, a tablet computer, a smartphone), and so on.

In some configurations, the server 752 is configured to perform the methods described in the present disclosure. For example, the processor 812 and memory 820 can be configured to perform the methods described herein.

In some configurations, data source 702 can include a processor 822, one or more data acquisition systems 824, one or more communications systems 826, and/or memory 828. In some configurations, processor 822 can be any suitable hardware processor or combination of processors, such as a CPU, a GPU, and so on. In some configurations, the one or more data acquisition systems 824 are generally configured to acquire data, images, or both, and can include an MRI system. Additionally or alternatively, in some configurations, the one or more data acquisition systems 824 can include any suitable hardware, firmware, and/or software for coupling to and/or controlling operations of an MRI system. In some configurations, one or more portions of the data acquisition system(s) 824 can be removable and/or replaceable.

Note that, although not shown, data source 702 can include any suitable inputs and/or outputs. For example, data source 702 can include input devices and/or sensors that can be used to receive user input, such as a keyboard, a mouse, a touchscreen, a microphone, a trackpad, a trackball, and so on. As another example, data source 702 can include any suitable display devices, such as an LCD screen, an LED display, an OLED display, an electrophoretic display, a computer monitor, a touchscreen, a television, etc., one or more speakers, and so on.

In some configurations, communications systems 826 can include any suitable hardware, firmware, and/or software for communicating information to computing device 750 (and, in some configurations, over communication network 754 and/or any other suitable communication networks). For example, communications systems 826 can include one or more transceivers, one or more communication chips and/or chip sets, and so on. In a more particular example, communications systems 826 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some configurations, memory 828 can include any suitable storage device or devices that can be used to store instructions, values, data, or the like, that can be used, for example, by processor 822 to control the one or more data acquisition systems 824, and/or receive data from the one or more data acquisition systems 824; to generate images from data; present content (e.g., data, images, a user interface) using a display; communicate with one or more computing devices 750; and so on. Memory 828 can include any suitable volatile memory, non-volatile memory, storage, or any suitable combination thereof. For example, memory 828 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 828 can have encoded thereon, or otherwise stored therein, a program for controlling operation of medical image data source 702. In such configurations, processor 822 can execute at least a portion of the program to generate images, transmit information and/or content (e.g., data, images, a user interface) to one or more computing devices 750, receive information and/or content from one or more computing devices 750, receive instructions from one or more devices (e.g., a personal computer, a laptop computer, a tablet computer, a smartphone, etc.), and so on.

In some configurations, any suitable computer-readable media can be used for storing instructions for performing the functions and/or processes described herein. For example, in some configurations, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present disclosure has described one or more preferred embodiments, and it should be appreciated that many equivalents, alternatives, variations, and modifications, aside from those expressly stated, are possible and within the scope of the invention.

1. A method for creating an automated system for determining disease states and conditions using magnetic resonance fingerprinting (MRF) data and magnetic resonance imaging (MRI) data acquired from a patient, the method comprising:
(a) accessing group MRF data acquired from a group of patients;
(b) accessing MRI data acquired from the group of patients;
(c) accessing annotated images, wherein the annotated images comprise bulk-pixel labels assigning bulk pixels in the annotated images to at least one tissue class, including a normal tissue class or a pathological tissue class; and
(d) training a machine learning system using the annotated images and the MRF data acquired from the group of patients using a patch-based approach to perform a pixel-based analysis of pixels outside the bulk-pixel labels to generate an automated system for determining disease states and conditions using a pixel-based machine learning system.

2. The method of claim 1, wherein to train the machine learning system, for a given pixel proximate to bulk-pixel labels of the at least one tissue class, the machine learning system analyzes the given pixel using the at least one tissue class of the proximate bulk-pixel label as ground truth.

3. The method of claim 1, wherein accessing the MRF data comprises:
accessing MRF time course data;
compressing the MRF time course data using singular value decomposition to represent the MRF time course data using a plurality of singular values; and
defining the MRF data based on the singular values.

4. The method of claim 1, further comprising randomly assigning each of the group of patients to one of k groups of which k−1 groups are defined as a training data set used to train the machine learning system.

5. The method of claim 4, further comprising repeating (d) a plurality of times with different groups of patients to generate the automated system for determining disease states and conditions using the pixel-based machine-learning system.

6. The method of claim 5, further comprising generating a probability map for the tissue class, wherein the probability map includes tissue classes assigned to pixels outside the bulk pixels in the annotated images.

7. The method of claim 1, wherein using the patch-based approach to perform the pixel-based analysis comprises training the machine learning using a 1 pixel×1 pixel×1 pixel patch of the MRF data.

8. The method of claim 1, wherein using the patch-based approach to perform the pixel-based analysis comprises training the machine learning using a patch that is larger than 1 pixel×1 pixel×1 pixel to account for spatial correlation in the MRF data.

9. A method for creating an automated system for generating improved annotations of magnetic resonance imaging (MRI) data of a patient using magnetic resonance fingerprinting (MRF) data, the method comprising:
accessing annotated MRI images for a group of patients, wherein the annotated MRI images contain a plurality of pixels, and wherein each of the plurality of pixels is labeled as an abnormal class or unlabeled;
accessing MRF data measured at each of the pixels labeled as an abnormal class for a first set of patients of the group of patients; and
training a machine learning algorithm using the MRF data wherein a ground truth is defined based on the labeled abnormal pixels.

10. The method of claim 9, further comprising accessing MRF data for a second set of patients of the group of patients at each of the unlabeled pixels and generating updated annotations by applying the machine learning algorithm to annotate each of the unlabeled pixels.

11. The method of claim 9, further comprising defining a surrounding patch of pixels for each of the plurality of pixels, and wherein training the machine learning algorithm is further based on MRF data measured at each pixel within the patch.

12. The method of claim 9, wherein accessing the MRF data comprises:
accessing MRF time course data;
compressing the MRF time course data using singular value decomposition to represent the MRF time course data using a plurality of singular values; and
defining the MRF data based on the singular values.

13. The method of claim 9, further comprising assigning each of the group of patients to one of k groups of which k−1 groups define a first set of patients used for training.

14. The method of claim 13, further comprising reassigning each of the group of patients to one of k groups of which k−1 groups define the first set of patients used for training and repeating:
accessing MRF data measured at each of the pixels labeled as abnormal for a first set of patients; and
training a machine learning algorithm based on the MRF data wherein a ground truth is defined based on the labeled abnormal pixels.

15. The method of claim 9, wherein the annotated MRI images are manually segmented.

16. An automated system for determining disease states and conditions using magnetic resonance fingerprinting (MRF) data and magnetic resonance imaging (MRI) data acquired from a patient, the system comprising a controller configured to:
receive reconstructed MRF data and MRI data acquired from the patient;
deliver reconstructed MRF data and the MRI data to a trained machine learning system, wherein the trained machine learning system was trained using MRF data acquired from a group of patients and annotated images acquired from the group of patients that comprise bulk-pixel labels assigning bulk pixels in the annotated images to at least one tissue class, including a normal tissue class or a pathological tissue class; and wherein the trained machine learning system performs a pixel-by-pixel analysis of the reconstructed MRI data to assign each pixel to a tissue class including at least a normal tissue class and a pathological tissue class; and
generate at least one machine-annotated image of the patient wherein each pixel in the at least one annotated image has an assigned tissue class.

17. The system of claim 16, further comprising delivering, to the trained machine learning system, an annotated image of the patient assigning bulk pixels to at least one of the normal tissue class or the pathological tissue class and, wherein the machine annotated image includes pixels that are reassigned by the trained machine learning system relative to the annotated image of the patient.

18. The system of claim 16, wherein the pixel-by-pixel analysis is a patch-based analysis in which assigning each pixel to a tissue class is based on a patch of MRF data surrounding each pixel.

19. The system of claim 16, wherein the MRF data comprises singular values produced by a singular value decomposition of an MRF time course at each pixel of the reconstructed MRI data.

20. A method for generating an automated system for cleaning annotated magnetic resonance imaging (MRI) images using magnetic resonance fingerprinting (MRF) data, the method comprising:
(a) accessing a plurality of annotated MRI images and corresponding MRF data, wherein each of the plurality of annotated images is defined by a first region and a second region, and wherein the first region comprises pixels labeled as abnormal and the second region comprises unlabeled pixels;
(b) splitting the annotated images into a first group of training data and a second group of test data; and
(c) using the training data to train a machine learning algorithm to assign pixel-wise labels to MRI images based on MRF data, wherein a ground truth used to train the machine learning algorithm is defined by the first region comprising the pixels labeled as abnormal of the training data.

21. The method of claim 20, further comprising:
(d) producing a labeled second region of each of the plurality of annotated MRI images by applying the machine learning algorithm to each of the second regions comprising the unlabeled pixels of the test data to label the second regions using MRF data; and
(e) combining each corresponding first region and labeled second region into a composite image to produce a cleaned annotated image for each of the annotated images.

22. The method of claim 21, further comprising re-splitting the annotated images into a new first group of training data and a new second group of test data and repeating (c) and (d) using the new first group and new second group a plurality of times to produce a plurality of cleaned annotated images.

23. The method of claim 22, further comprising producing a plurality of probability maps based on the plurality of cleans annotated images.

24. The method of claim 23, further comprising:
thresholding the plurality of probability maps to create new binary annotated images; and
training a second machine learning algorithm using the new binary annotated images as a ground truth.
